(19) United States
(12) Patent Application Publication
(10) Pub. No.: US 2025/0114635 A1
(43) Pub. Date: Apr. 10, 2025

(54) OPTICAL NEURAL PROBE WITH DUAL COLOR LED

(71) Applicant: UNIVERSITY OF MASSACHUSETTS, Westborough, MA (US)

(72) Inventor: Guangyu Xu, Amherst, MA (US)

(21) Appl. No.: 18/908,922

(22) Filed: Oct. 8, 2024

Related U.S. Application Data
(60) Provisional application No. 63/543,402, filed on Oct. 10, 2023.

(51) Int. Cl. A61N 5/06

(52) U.S. Cl. CPC A61N 5/0622; A61N 5/0601; A61N 2005/0612; A61N 2005/0632; A61N 2005/0652; A61N 2005/0663

(57) ABSTRACT

An optical probe includes a probe body and a shank extending from the probe body to a tip. The tip includes a first plurality of light emitting diode pixels and a first plurality of recording electrodes. The first plurality of light emitting diode pixels includes a first light emitting diode and a second light emitting diode, wherein the first light emitting diode and the second light emitting diode emit a different color of light. The first plurality of light emitting diode pixels are arranged in groups. The light emitting diode pixels and groups of pixels are arranged in a particular manner. Methods for the manufacture of the probe and methods of using the probe are also described.

### CROSS-REFERENCE TO RELATED APPLICATION

This application claims priority to U.S. Provisional Patent Application No. 63/543,402, filed Oct. 10, 2023, the contents of which are hereby incorporated by reference in their entirety.

### FEDERAL RESEARCH STATEMENT

This invention was made with government support under Grant Nos. 1835268, 2055457, and 2046031, each awarded by the National Science Foundation. The government has certain rights in the invention.

### BACKGROUND

Optogenetics, a method to optically control cell activity via light-sensitive proteins, has risen to the focus of the neuroscience community for its combined advantages on temporal precision, cell-type specificity, and bi-directionality (i.e., enhance or suppress neural activity upon the color of light). The capability of bi-directional neuromodulation not only offers an alternative approach to dissect heterogenous brain circuits but also holds promise to develop therapeutic interventions (e.g., inhibiting epilepsy). Yet, the dual-colored light required for bidirectional optogenetic control of the same neurons (colocalized modulation) often occupies a broad optical bandwidth hindering its capability to co-work with fluorescent live cell imaging to examine optogenetic effects in a low-crosstalk manner. For instance, microscopy or fiberscope based all-optical approaches were often to combine calcium/voltage imaging with single-colored (unidirectional) optogenetic control of the cells. In this perspective, optogenetic electrophysiology, a powerful approach that shares the benefits of high-resolution electrical recording and high-precision optical manipulation of cells, is possible to circumvent the bandwidth limitation of all-optical methods and trace real-time brain dynamics under bi-directional optogenetic patterns with low cross-talk.

Accordingly, there remains a need for improved implantable neural interfaces. It would be particularly advantageous to provide neural interfaces that can output different colors of light with high spatial resolutions and include arrays of microelectrodes (i.e., MEA) for neural recording, which could be used to precisely map the functional circuits in the brain, and ultimately develop animal disease models.

### SUMMARY

An optical neural probe comprises a probe body; a shank extending from the probe body to a tip; wherein the tip comprises a first plurality of light emitting diode pixels and a first plurality of recording electrodes, wherein the first plurality of light emitting diode pixels comprises a first light emitting diode and a second light emitting diode, wherein the first light emitting diode and the second light emitting diode emit a different color of light; wherein the first plurality of light emitting diode pixels are arranged in groups, wherein each light emitting diode pixel in a group has a center-to-center distance of 10 to 50 micrometers; and wherein each group of light emitting diode pixels has a center-to-center distance of 30 to 150 micrometers.

A method for the manufacture of an optical neural probe comprising a probe body; a shank extending from the probe body to a tip; wherein the tip comprises a first plurality of light emitting diode pixels and a first plurality of recording electrodes, wherein the plurality of light emitting diode pixels comprises a first light emitting diode and a second light emitting diode, wherein the first light emitting diode and the second light emitting diode emit a different color of light; wherein the method comprises contacting a first layer comprising a first light emitting diode precursor with a substrate; patterning the first light emitting diode precursor to provide a first light emitting diode array; applying a first passivating layer to the first light emitting diode array; contacting a second layer comprising a second light emitting diode precursor with the first passivating layer; patterning the second light emitting diode precursor to provide a second light emitting diode array; applying a second passivating layer to the second light emitting diode array; patterning a microelectrode array on the second passivating layer; applying a third passivating layer to the microelectrode array; and etching the substrate to provide the optical neural probe having preselected dimensions.

A method of stimulating target neurons expressing light responsive proteins in a tissue, wherein the method comprises inserting an optical neural probe into the tissue; activating the optical neural probe to deliver light to stimulate the target neurons; and measuring neuronal activity with the recording electrodes in response to stimulating the target neurons.

The above described and other features are exemplified by the following figures and detailed description.

### BRIEF DESCRIPTION OF THE DRAWINGS

The following figures represent exemplary embodiments.

FIG. 1 shows an exemplary optical neural probe tip according to an aspect of the present disclosure.

FIG. 2 shows a schematic illustration of a probe being inserted into the mouse somatosensory cortex, wherein the light emitting diodes and microelectrode array are positioned across layers IV and V, and surrounded by excitatory (blue triangle) and/or inhibitory (red/green oval) neurons.

FIG. 3 shows a schematic illustration of a method of making the optical neural probe according to an aspect of the present disclosure.

FIG. 4 shows results from in vivo bi-directional optogenetic electrophysiology across layers IV and V, including peristimulus time histograms (PSTH) summed from all 112trials (16 active electrodes with 7 trials each) (left), spike count across the microelectrode array (center), and percentage changes of the spike count compared to those from the [−100 ms, 0] window (before LED pulsing) (right).

FIG. 5 shows positions of illuminated light emitting diode pixels and whisker stimulations, raster plots of 7 trials from a representative electrode, where the green windows represent the whisker pulses, and the red or blue windows represent the red or blue light emitting diode pulses, PSTH summed from all 112 trials (16 active electrodes with 7 trials each), spike count across the microelectrode array, and percentage changes of the spike count in compared to the whisker-stimulation-alone experiment.

### DETAILED DESCRIPTION

It is desirable to provide neural interfaces that can output different colors of light with high spatial resolutions and include arrays of microelectrodes (i.e., MEA) for neural recording, which could be used to precisely map the functional circuits in the brain, and ultimately develop animal disease models. To date, a variety of optoelectronic neural probes assembled with light sources (either passive or active ones) and microelectrodes have been applied to optogenetically modulate neural activities at various regions of the brain. Among them, single-colored micro-LED-based probes were noted for their spatial resolution, scalability, and power consumption, and have been optimized to minimize their photovoltaic artifact on neural recording. Yet, limited by feasible integration methods, probes with high density arrays of dual-color light sources are still missing, making high-resolution bi-directional in vivo optogenetic electrophysiology technically challenging. This is a non-trivial task as it requires monolithic integration of different types of active light materials that can be patterned into a small footprint adjacent to low-artifact microelectrodes, all of which need to be highly scalable and compatible with the following deep etching steps to form a sub-100 μm-thick shank.

The present inventor has found that the foregoing technical challenges can be addressed by a neural probe monolithically integrated with close-packed dual-color micro-LEDs and electrodes (e.g., in 20 and 50 μm pitches, respectively). Both blue and red LEDs feature high brightness, small spot size, fast response, and low voltage operation, whereas the microelectrodes are coated with a poly (3,4-ethylenedioxythiophene) polystyrene sulfonate (PEDOT:PSS) layer for high-fidelity neural recording. The resulting high-yield devices demonstrated their use for bidirectional in vivo optogenetic electrophysiology across layers IV and V of mouse somatosensory cortex, where dual-color LEDs were observed to excite and inhibit brain dynamics in a cortical-layer-specific manner, possibly due to interlayer signaling pathways. Such layer-specific bidirectional in vivo optogenetic studies can add to high-resolution interrogation of the brain circuitry and ultimately shed light on neurological disease models. A significant advantage is therefore provided by the present disclosure.

Accordingly, an aspect of the present disclosure is an optical neural probe. The optical neural probe according to the present disclosure comprises a probe body and a shank extending from the probe body to a tip. The optical neural probe can be used to optically stimulate or silence neurons and record electric responses to the stimulus.

The tip of the probe is located furthest from the probe body and comprises a first plurality of light emitting diode pixels and a first plurality of recording electrodes. The tip of the probe may be tapered. The first plurality of light emitting diode pixels comprises a first light emitting diode pixel and a second light emitting diode pixel. The first light emitting diode pixel and the second light emitting diode pixel emit a different color of light (i.e., each emit a different wavelength of light). The light can be visible or non-visible light.

Light emitting diode pixels can comprise a group III-V semiconductor such as GaN, InP, AlGaN, and the like. Such materials can be deposited on a sapphire, silicon, or other substrate through molecular beam epitaxy, chemical vapor deposition, or other deposition technique. In an aspect, the first light emitting diode pixel can be a red light emitting diode pixel (i.e., emitting light having a wavelength of 625 to 740 nanometers, or 625 to 700 nanometers, or 726 to 675 nanometers), and the second light emitting diode pixel can be a blue light emitting diode pixel (i.e., emitting light having a wavelength of 400 to 525 nanometers, or 400 to 500 nanometers, or 450 to 500 nanometers). Other colors (i.e., wavelengths of light) can be used and can be suitably selected depending on the target neuron for the optical probe, guided by the present disclosure. In a specific aspect, the red light emitting diode pixel can comprise AlGaInP. In a specific aspect, the blue light emitting diode pixel can comprise GaN.

The first plurality of light emitting diode pixels are arranged in groups. Preferably, each group can comprise at least one first light emitting diode pixel and at least one second light emitting diode pixel. Each light emitting diode pixel in the group is arranged such that a center-to-center distance between each pixel is in the range of 10 to 50 micrometers, for example 10 to 40 micrometers, or 10 to 30 micrometers, or 12 to 28 micrometers, or 15 to 25 micrometers, or 18 to 22 micrometers. Each group of light emitting diode pixels are arranged such that a center-to-center distance between each group is in the range of 30 to 150 micrometers, for example 30 to 125 micrometers, or 30 to 100 micrometers, or 30 to 85 micrometers, or 35 to 65 micrometers, or 40 to 60 micrometers, or 45 to 55 micrometers.

In an aspect, the light emitting diode pixels in each group can be arranged in a grid pattern. For example, when a group comprises two of the first light emitting diode pixels and two of the second light emitting diode pixels, the pixels can be arranged in a two by two pixel grid. Preferably, the two first light emitting diode pixels are positioned adjacent to each other, and the two second light emitting diode pixels are positioned adjacent to each other.

The plurality of light emitting diode pixels can generally be arranged in any suitable number of groups. For example, the first plurality of light emitting diode pixels can comprise at least one group of light emitting diode pixels, and up to, for example 25 groups of light emitting diode pixels. In an aspect the first plurality of light emitting diode pixels can comprise 2 to 20 groups, or 2 to 15 groups, or 5 to 10 groups. In an aspect, the plurality of light emitting diode pixels can comprise two, three, four, five, six, seven, eight, nine, or ten groups of light emitting diode pixels, wherein each group comprises at least one first light emitting diode pixel and at least one second light emitting diode pixel, preferably wherein each group comprises two of the first light emitting diode pixels and two of the second light emitting diode pixels.

The light emitting diode pixels of the first plurality of light emitting diode pixels can have length of 3 to 10 micrometers, or 5 to 10 micrometers, or 6 to 8 micrometers, and a width of 3 to 10 micrometers, or 5 to 10 micrometers, or 6 to 8 micrometers. The light emitting diode pixels can have a height (i.e., from the substrate surface) of 2 to 10 micrometers, or 3 to 7 micrometers, or 5 to 6 micrometers.

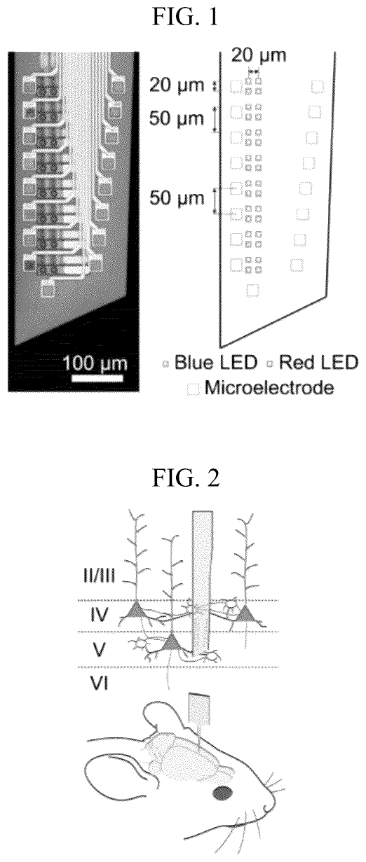

The tip further comprises a first plurality of recording electrodes, also referred to herein as a microelectrode array (MEA). In an aspect, the recording electrodes are disposed adjacent to the plurality of light emitting diode pixels. In an aspect, the plurality of light emitting diode pixels can be surrounded recording electrodes. An exemplary arrangement of the light emitting diode pixels and the recording electrodes on the tip of the probe can be as shown in FIG. 1. In an aspect, the recording electrodes can be arranged in an array which is generally positioned at the periphery or edge of the tip. The electrodes can serve to receive an electrical response from a neuron upon stimulation by the light emitted from the light emitting diode. Materials suitable for the electrodes are generally known, and can comprise for example chromium, gold, iridium, platinum, other metals, an oxide thereof, or carbon nanotubes. In an aspect, the recording electrodes can comprise a chromium-gold alloy. Each recording electrode can have a length of 15 to 25 micrometers and a width of 15 to 25 micrometers.

In an aspect, the plurality of light emitting diode pixels can span a distance of 300 to 1500 micrometers at the probe tip. For example, in an aspect, the plurality of light emitting diode pixels can span a distance of 300 to 450 micrometers. In another aspect, the plurality of light emitting diode pixels can span a distance of 1000 to 1500 micrometers.

In an aspect, the optical neural probe can optionally further comprise a second plurality of light emitting diode pixels and a second plurality of recording electrodes. The second plurality of light emitting diode pixels and second plurality of recording electrodes can be arranged as described for the first plurality of light emitting diode pixels and recording electrodes. For example, the second plurality of light emitting diode pixels can comprise a first light emitting diode pixel and a second light emitting diode pixel, wherein the first light emitting diode pixel and the second light emitting diode pixel can be as described above. In particular, the first light emitting diode pixel and the second light emitting diode pixel emit a different color of light.

The second plurality of light emitting diode pixels can be arranged in groups. Preferably, each group can comprise at least one first light emitting diode pixel and at least one second light emitting diode pixel. Each light emitting diode pixel in the group can be arranged such that a center-to-center distance between each pixel is in the range of 10 to 50 micrometers, for example 10 to 40 micrometers, or 10 to 30 micrometers, or 12 to 28 micrometers, or 15 to 25 micrometers, or 18 to 22 micrometers. Each group of light emitting diode pixels can be arranged such that a center-to-center distance between each group is in the range of 30 to 150 micrometers, for example 30 to 125 micrometers, or 30 to 100 micrometers, or 30 to 85 micrometers, or 35 to 65 micrometers, or 40 to 60 micrometers, or 45 to 55 micrometers.

The light emitting diode pixels of the second plurality of light emitting diode pixels can have length of 3 to 10 micrometers, or 5 to 10 micrometers, or 6 to 8 micrometers, and a width of 3 to 10 micrometers, or 5 to 10 micrometers, or 6 to 8 micrometers. The light emitting diode pixels can have a height (i.e., from the substrate surface) of 2 to 10 micrometers, or 3 to 7 micrometers, or 5 to 6 micrometers.

In an aspect, the light emitting diode pixels in each group of the second plurality of light emitting diode pixels can be arranged in a grid pattern. For example, when a group comprises two of the first light emitting diode pixels and two of the second light emitting diode pixels, the pixels can be arranged in a two by two pixel grid. Preferably, the two first light emitting diode pixels are positioned adjacent to each other, and the two second light emitting diode pixels are positioned adjacent to each other.

The second plurality of light emitting diode pixels can generally be arranged in any suitable number of groups. For example, the second plurality of light emitting diode pixels can comprise at least one group of light emitting diode pixels, and up to, for example 25 groups of light emitting diode pixels. In an aspect the second plurality of light emitting diode pixels can comprise 2 to 20 groups, or 2 to 15 groups, or 5 to 10 groups. In an aspect, the second plurality of light emitting diode pixels can comprise two, three, four, five, six, seven, eight, nine, or ten groups of light emitting diode pixels, wherein each group comprises at least one first light emitting diode pixel and at least one second light emitting diode pixel, preferably wherein each group comprises two of the first light emitting diode pixels and two of the second light emitting diode pixels.

The second plurality of recording electrodes can be disposed adjacent to the second plurality of light emitting diode pixels. In an aspect, the second plurality of light emitting diode pixels can be surrounded recording electrodes.

The second plurality of light emitting diode pixels and recording electrodes can be positioned at a distance from the first plurality of light emitting diode pixels and recording electrodes. For example, in an aspect, the second plurality of light emitting diode pixels and recording electrodes can be positioned 1 to 2 millimeters away from the first plurality of light emitting diode pixels and the first plurality of recording electrodes.

Thus in an aspect, the optical neural probe can be considered to include two light emitting sites (i.e., one being the first plurality of light emitting diode pixels at the tip of the probe, and the second being the second plurality of light emitting diode pixels along the shank of the probe), thereby allowing simultaneous neuron stimulation at multiple stimulation zones from a single optical neural probe, along with the collection of electrical responses at corresponding multiple positions along the shank. Each stimulation zone represents a region from which neural electrical responses to stimulation or silencing are desired to be recorded or a region that receives sufficient neuron-affecting light to stimulate or silence neurons. In this aspect, each stimulation zone at least partially overlaps one of the array regions (i.e., one of the first or second plurality of light emitting diode pixel arrays) by virtue of the position of each light-emitting site in relation to each stimulation zone.

The shank of the optical neural probe can be configured to locate the tip of the probe comprising the plurality of light emitting diode pixels at a probing depth to target a neuron. In an aspect, the shank can have a thickness of less than 100 micrometers, preferably a thickness of 40 to 80 micrometers; a width of 100 to 250 micrometers; and a length of 3 to 12 millimeters. Suitable materials for the shank of the optical probe are generally known, and for example the shank can comprise silicon or an alloy thereof.

Electrode leads extend along the shank to electrically connect each electrode and each light emitting diode pixel to the probe body. The electrode leads can transmit electrical responses received from the recording electrodes from stimulated neurons to the probe body. In an aspect, leads can be, for example, up to 0.5 micrometers in thickness (i.e., raised from the surface of the shank) and 1 to 5 micrometers in width. The probe body can comprise electrical connections for electrical communication with a printed circuit board. The electrical response can be sent through electrical connections to the printed circuit board.

In an aspect, the optical neural probe can optionally further comprise a second shank extending from the probe body to a second tip. The second shank can, in an aspect, be a duplicate of the first shank, with the pluralities of light emitting diode pixels and recording electrodes being substantially identical to those on the first shank. For example, the second tip of the second shank can comprise a third plurality of light emitting diode pixels and a third plurality of recording electrodes. In an aspect, the third plurality of light emitting diode pixels can be the same as the first plurality of light emitting diode pixels. The third plurality of light emitting diode pixels comprises a third light emitting diode and a fourth light emitting diode, wherein the third light emitting diode and the fourth light emitting diode emit a different color of light (e.g., red and blue light). The light emitting diode pixels can be arranged as described above. For example, the third plurality of light emitting diode pixels can be arranged in groups, wherein each group of light emitting diode pixels can have a center-to-center distance of 30 to 150 micrometers, and each light emitting diode pixel within the third plurality of light emitting diode pixels has a center-to-center distance of 10 to 50 micrometers. The third plurality of light emitting diode pixels can similarly comprise 1 to 25 groups of light emitting diode pixels.

The optical neural probe of the present disclosure can be made by a method which advantageously allows for the close packed arrays of multi-color light emitting diode pixels and microelectrodes. A method for the manufacture of the optical neural probe represents another aspect of the present disclosure. The method comprises contacting a first epilayer comprising a first light emitting diode precursor with the tip. The term epilayer as used herein refers to an epitaxially grown layer. The method further comprises patterning the first light emitting diode precursor to provide a first light emitting diode pixel array, and applying a first passivating layer to the first light emitting diode pixel array. A passivation layer refers to a layer which can render the underlying components “passive”, or less affected by a future processing step or working conditions, and can therefore prevent undesired degradation. Thus the passivation layer is generally inert. The first passivating layer can comprise any passive material generally known in the art, provided that they are optically transparent, particularly with respect to the wavelengths of light selected for the light emitting diode pixels of the probe. In an aspect, the passivating layer can comprise an epoxy, for example an epoxy-containing negative photoresist capable of being crosslinked upon exposure to ultra-violet light. An exemplary epoxy material is known as SU-8, a bisphenol A novolac epoxy having eight epoxy groups.

The method further comprises contacting a second epilayer comprising a second light emitting diode precursor with the first passivating layer; patterning the second light emitting diode precursor to provide a second light emitting diode pixel array; and applying a second passivating layer to the second light emitting diode pixel array. The second passivating layer can be the same as the first passivating layer. The method further comprises patterning a microelectrode array on the second passivating layer; applying a third passivating layer to the microelectrode array; and etching the probe to provide the optical probe having preselected dimensions. The etching can be by deep reactive ionized etching. The method of manufacturing the optical neural probe can be as shown in FIG. 2. The optical probe shank can be bound to a printed circuit board, and subsequently encapsulated prior to use. Encapsulation can be with, for example, a UV-curable epoxy.

Another aspect of the present disclosure is a method of stimulating a target neuron expressing a light responsive protein in a tissue. The method can comprise inserting an optical neural probe into the tissue. The optical probe can be as described above. The method can further comprise activating the optical neural probe to deliver light to stimulate the target neurons, and measuring the response as a result of stimulating the neurons. Activating the optical neural probe can comprise activating at least one light emitting diode pixel (i.e., causing the light emitting diode pixel to emit light, for example by applying an electrical current. The response can be measured by the plurality of recording electrodes, which can transmit the detected signals to an external system for monitoring the signals (e.g., a computer system).

In an aspect, the tissue can be a test subject such as a living mammal, for example an animal or human. It is noted that the dimensions of the optical probe can be adjusted accordingly to suit the size of the test subject. For example it will be understood that a mouse test subject may require a smaller probe than for a larger animal (e.g., a monkey) or a human. The skilled person guided by the present disclosure will understand how to scale up the optical neural probe described herein to suit the intended test subject.

In an aspect, the tissue can be an organoid, for example a brain organoid or a neural organoid. The term “organoid” as used herein refers to a tissue formed in vitro from a collection of cells having a cellular organization and gross morphology similar to that of the tissue of origin for at least a subset of the cells in the collection. By “in-vivo-like gross and cellular morphology of a tissue of interest” is meant a three-dimensional shape and cellular organization substantially similar to that of the tissue or a component of the tissue in vivo. By “substantially similar to that of the tissue in vivo” is meant that the structure is visibly identical or similar to (for example in terms of morphology or the expression of appropriate marker proteins) or functionally similar to the structure (for example, expresses at least 5% of a marker protein of the native form of the tissue, produces at least 5% of the amount of a protein produced by the structure, or performs an enzymatic reaction at a level that is at least 5% of the level of reaction performed by the tissue). The organoid may be derived from stem cells.

This disclosure is further illustrated by the following examples, which are non-limiting.

### EXAMPLES

### Probe Structure Design

In bi-directional optogenetic studies, the activation spectra of excitatory and inhibitory opsins need to be well separated from each other to minimize their optical crosstalk. For this reason, here Chrimson39 and GtACR240 were chosen as excitatory and inhibitory opsins, respectively, which have shown their low crosstalk in bi-directional optogenetic experiments. Furthermore, these two opsins have been fused together into AAV-BiPOLES vectors, allowing both of them to be delivered to the same cortical neurons by single AAV-virus injection. To optically activate GtACR2 and Chrimson, on the hardware side GaN-and AlGaInP-based micro-LEDs (i.e. blue and red LEDs) were built, which can output 462/19 nm and 625/10 nm light that fall in the activation spectra of these two opsins, respectively. This approach of building dual-color LEDs is intended to leverage commercial epitaxial GaN-on-Si and AlGaInP-on-GaAs wafers, which offer bright, stable, and scalable LED arrays.

While blue and red LEDs can be separately built in a high-density array form, integrating both of them monolithically on the same probe is the key for high-resolution bi-directional optogenetic studies. Here this was achieved by physically transferring the epitaxial layers (i.e., epilayer) of an AlGaInP-on-GaAs wafer on top of the GaN-on-Si wafer, with a SU8 film placed in between to bond these epilayers. These heterogeneously stacked epilayers readily lend themselves to high yield fabrication of red LEDs on top of blue LEDs, allowing for their monolithic integration.

On the neural recording end, the MEA needs to lower its electrode impedance to obtain a high signal-to-noise-ratio (SNR) during optogenetic experiments. For this reason, an Au-based MEA electroplated with a PEDOT:PSS layer was fabricated, which can effectively reduce the electrode impedance. This electroplating step reduces the electrode impedance across the entire MEA.

Finally, to allow for high-resolution optogenetic electrophysiology across cortical layers, both dual-color LEDs and microelectrodes were built in close-packed arrays along different access depths of the probe. For this reason, the neural probe is designed to have 8 groups of dual color LEDs closely packed in a 50 μm vertical pitch. Each group is composed of 2 blue and 2 red LEDs placed in a 20 μm pitch, with all LEDs being 7 μm-by-7 μm in size. A total of 17 recording electrodes with 20 μm-by-20 μm in size are placed next to these 8 groups of dual-color LEDs, as shown in FIG. 1. The resulting LEDs and electrodes cover about a 380 μm vertical scan at the probe tip, which was designed to conduct optogenetic electrophysiology across different cortical layers in the mouse brain, as shown in FIG. 2.

### Fabrication and Packaging of the Probe

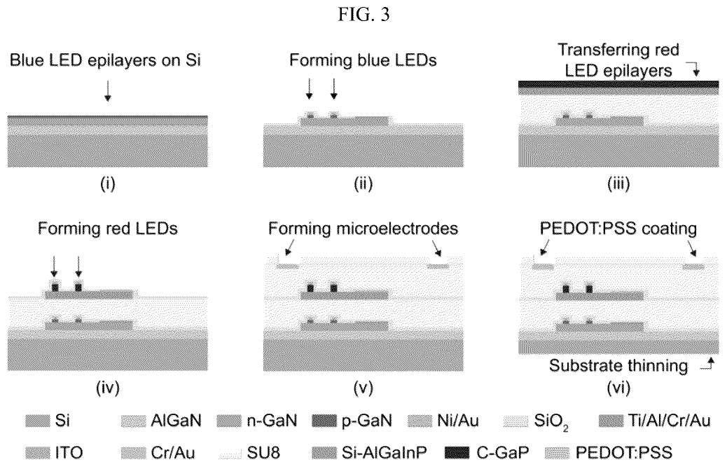

A schematic illustration of the fabrication process is shown in FIG. 3. Specifically, probes were fabricated by patterning an 8-by-2 cross-barred blue micro-LED array on a GaN-on-Si wafer. These blue LEDs are placed in a horizontal/vertical pitch of 20/50 μm, contacted by a Ni/Au current spreading layer to lower their turn-on voltage, and passivated by a plasma-enhanced chemical vapor deposition (PECVD) based SiO₂ layer. The resulting array was spin coated with a SU8 layer, followed by a soft baking step, which later serves to bond the epilayers from the AlGaInP-on-GaAs wafer.

On the other end, wet etching was applied to remove the GaAs substrate from AlGaInP-on-GaAs wafer, leaving 5-6 μm thick epilayers (consist of AlGaInP-based quantum well layers) floating in the etchant. A pipette was used to suck the epilayers out of the etchant, rinsed them with DI water, and placed them on the soft-baked SU8 layer coated on blue LEDs. Afterwards, the resulting device sequentially went through a ca. 20 min drying step in a desiccator, a UV exposure step to cross-link the SU8, and a 30 mins hard-baking step for permanent bonding of the epilayers. Importantly, such heterogeneously stacked epilayers allow us to pattern an 8-by-2 cross-barred red LED array using the same approach as we used to form the blue LED array, with the red and blue LED pixels being closely packed to each other. The resulting dual-color LEDs were passivated with a 5 μm thick SU8 layer, which later serves to lessen the electromagnetic interference (EMI) of the neural recording traces.

On top of the passivated dual-color LEDs, an Au-based MEA was patterned and passivated with a stack of SiO₂ (by sputtering) and SU8 layers. The resulting electrodes are 20 μm-by-20 μm in size and placed with a vertical pitch of 50 μm on both sides of the dual-color LEDs. Similar to the approach used to form silicon neural probes, deep reactive ionized etching (DRIE) steps were applied from both the front and back sides of the device to define the horizontal dimension and the thickness of a shank structure, respectively. The resulting shank (contains dual-color LEDs and the MEA) is about 200 μm in width, 5 mm in length, and 50 μm in thickness, representing the completion of the monolithic probe fabrication.

Lastly, the as-made shank was wire-bonded onto a printed circuit board (PCB) and encapsulated with UV-curable epoxy. These packaging steps allow for electroplating the MEA with a PEDOT:PSS layer, which prepares the probes for subsequent experimental use.

### Optoelectronic Performance of the Neural Probe

To examine if the packaged probes suffice in vivo bi-directional optogenetic electrophysiology studies, the optoelectronic performance of the dual-color LEDs and the PEDOT:PSS-coated MEA was characterized.

To enable in vivo optogenetic control at cellular levels, both blue and red LEDs are required to output bright, localized, and fast-switching light, ideally in a low-voltage operation. To this end, the optical power density (P_{blue/red}) and the spatial profile of the illumination spot (I_{light}) of each LED pixel was measured. When biased at an injection current (I_{LED}) of 9 [20] μA, blue [red] LED pixels readily show high brightness with P_{blue/red}˜4 mW/mm², which falls into the range required for in vivo optogenetic control over neural activity via GtACR2 [Chrimson]. While the difference of I_{LED} between blue and red LEDs did exist (likely due to different epilayers and contact resistances), it is encouraging to note that such high brightness is achieved with the driving voltage across each pixel (V_{LED}) being about 3.5 V [7 V] for blue [red] LEDs. Such low-voltage operation leads to 31.5 [140] μW electrical power dissipation per blue [red] LED pixel, which has been deemed suitable for in vivo use. Moreover, it was noted that the full width at half maximum (FWHM) of the LED light spot is typically about 10 μm at the probe surface with I_{LED} ranging from 2 to 10 [5 to 25] μA in blue [red] LEDs. Such localized light output is essential for bi-directional optogenetic control over individual neurons, whose soma are 10˜20 μm in size. Finally, it was found that such bright, localized pixel output can be pulsed with a 10 ms-duration at up to 40 Hz pulsing frequencies. Each pulse featured a 2 ms rising and falling times, suitable for temporally precise neurostimulation. These results suggest that the dual-color LEDs meet the brightness, resolution, and speed requirement for in vivo bi-directional optogenetics at cellular levels.

To assess the performance of our MEA for high-SNR recording, electrochemical impedance spectrometry (EIS) and cyclic voltammetry (CV) testings of all 17 electrodes was conducted. The results show that the PEDOT:PSS layer was effective in altering the EIS (from 0.1 to 100 kHz) and CV (from −0.6 to 0.8 V) data measured from the MEA. First, after the electroplating step, all 17 electrodes dropped their EIS impedance amplitude at 1 KHz from 3.07±0.55 MΩ to 44±3.76 kΩ (although non-uniform coating on some electrodes may occur), and increased their EIS phase in the entire frequency range. This result suggests that the PEDOT:PSSlayer effectively reduced the electrode impedance, and changed the electrodes to be less capacitive (i.e., away from −90° phase). Second, the PEDOT:PSS coating step increased the current values in the CV curves by about one order with no apparent redox peaks. This result suggests that the PEDOT:PSS layer effectively augmented non-Faradaic charging processes, likely because its roughness and porosity increased the effective surface area of the electrode, and thus decreased the electrode impedance. For these reasons, the low-impedance, non-Faradaic MEA lends itself to high-SNR in vivo neural recordings.

### Suppressing the Recording Artifact

Before performing in vivo optogenetic studies, whether the low-impedance MEA could feature low recording artifact for the sake of high signal-to-background ratio (SBR) was examined. In fact, SNR and SBR have been recognized as two equally important figures-of-merits to achieve high-fidelity neural recording. Furthermore, the recording artifact has been concerning in monolithically integrated neural probes, where the neural recording could be impacted by EMI-induced, photovoltaic-induced, or residual artifacts. For these reasons, it is essential to evaluate if the pulsing of LEDs (required for optogenetic testing) will cause non-negligible artifacts in the recording traces.

To quantify the amplitude of the recording artifact, the probe was immersed in 1×Dulbecco's phosphate-buffered saline (DPBS) solution, and applied a single 50 ms voltage pulse to transiently turn on and off single LED (V_{high} is ca. 3.5 V [7 V] in blue [red] LED). Such pulsing operation of LEDs was intended to emulate the illumination pattern and light brightness used in optogenetic studies. It fluctuated the voltage traces of all 17 electrodes that were bandpass (BP) filtered at 250 Hz-10 kHz, mainly near the rising/falling edges of the pulse with a duration of <4 ms. The mean peak-to-peak magnitude (Vpp) of these traces is taken as the artifact amplitude.

Two strategies were examined to mitigate the recording artifact. First, a 0.1 μF capacitor was added in parallel to the output of the source-measurement unit (SMU) used to pulse the LEDs. This approach serves to low-pass (LP) filter the voltage pulses and slows down the LED switching (from 50 μs to 2.5 ms). The resulting artifact amplitude was significantly reduced by 90%, likely due to the removal of high-frequency artifacts. Second, with this LP capacitor being connected, the off-state voltage of the LEDs (V_{low}) was elevated from 0 to the near-threshold value of the select LEDs (2.5 V [3 V] for blue [red] LEDs). This approach serves to cut the voltage ramping on LEDs and reduce the capacitive coupling to recording traces, resulting in an additional ca. 10% drop of the artifact amplitudes. Together, such LP filtered, V_{low} adjusted pulsing of blue [red] LEDs readily leads to 100 [200] μV of artifact with sub-2 ms duration, which is on par with other LED-integrated neural probes used for optogenetics. Importantly, such low-level, short-duration artifact does not overwhelm the typical amplitude of the neural spikes, and can be simply removed from the traces for artifact-free analysis. Taking one step further, the data show that simultaneously illuminating more LED pixels does not necessarily increase the artifact amplitude. This result suggests that the recording artifact largely comes from EMI across leading wires rather than the photovoltaic effect.

It was also noted that driving LEDs with a sinusoidal voltage waveform can further reduce the artifact. This approach however is unable to keep LEDs at their maximum P_{blue/red} in the entire pulse window. LP-filtered square pulses were therefore selected to drive LEDs within this work.

### Bi-Directional in Vivo Optogenetic Electrophysiology

After in vitro testing of the probe performance and the recording artifact, the dual-color neural probes were applied to the cortex layers of anesthetized mice to assess their capabilities for bi-directional in vivo optogenetic electrophysiology. Specifically, AAV-BiPOLES-CaMKII vectors were injected to the cortical regions of the mice, which allows Chrimson and GtACR2 opsins to express in the excitatory neuron populations across the primary somatosensory cortex. Followed by 3 weeks of recovery and viral expression, the neural probe was inserted into the brain of head-fixed, anesthetized mice, with the LEDs and electrodes being located across layers IV and V in the SI region (examined by the brain slice image taken after in vivo testing). These two layers were chosen since they play vital roles in forming the circuit pathways of sensory perception and controlling the sensory responses. Since each layer spans 200-300 μm in depth, they can be accessed separately by illuminating either the top or the bottom 3 rows of LEDs (with a total of 380 μm vertical span) on the probe, and record the neural activity in both layers using the MEA.

Bi-directional optogenetics electrophysiology was conducted by sequentially pulsing 6 red and 6 blue LEDs located in layer V (red [blue]: seven 2-s periods with toff/on/off=0.5/0.05/1.45 s [t_{off/on/off/on}=0.5/0.5/0.5/0.5 s]; blue LEDs were pulsed with a longer tₒₙ to enhance the inhibition effect), with the MEA traces being BP-filtered at 250 Hz-10 kHz to detect neural spiking events; each 2-s period is defined as a trial here. To conduct artifact-free analysis of the recorded traces, the 4-ms of data centered at the rising/falling edges of each LED pulse was excluded. The rest of the traces were then used to extract spikes whose negative amplitudes are >5 times the noise floor. Next, for each electrode, spikes recorded in 7 trials were pooled for principal component analysis (PCA), and applied density-based spatial clustering of applications with noise (DBSCAN) to sort spikes. It is noted that the sorted spike waveforms (excluding noises) were often with a sub-500 μs trough-to-peak duration, suggesting their possible origins from fast-spiking interneurons near the MEA (e.g., parvalbumin-expressing (PV) neurons). Such interneuron activities may be evoked by optogenetically controlled excitatory neurons (opsins were expressed in excitatory neurons via the CaMKII promoter) via local circuit. The activity of excitatory neurons (typically with >500 μs durations), on the contrary, was not as significant here, likely because they were further distance away from the MEA or in turn inhibited by connected interneurons.

To quantify the optogenetic effect of pulsed LEDs, the spike timing during seven 200-ms windows was plotted, one from each 2-s trial, followed by summing spike count from 16 electrodes (one electrode failed in experiments) in these 7 windows to yield peristimulus time histograms (PSTH). Moreover, spike count recorded at each electrode within [0, 100 ms] and [−100 ms, 0] windows was compared to examine LED-induced optogenetic effects across the MEA. After in vivo recording, brain slices were collected to examine the probe position across cortical layers by imaging.

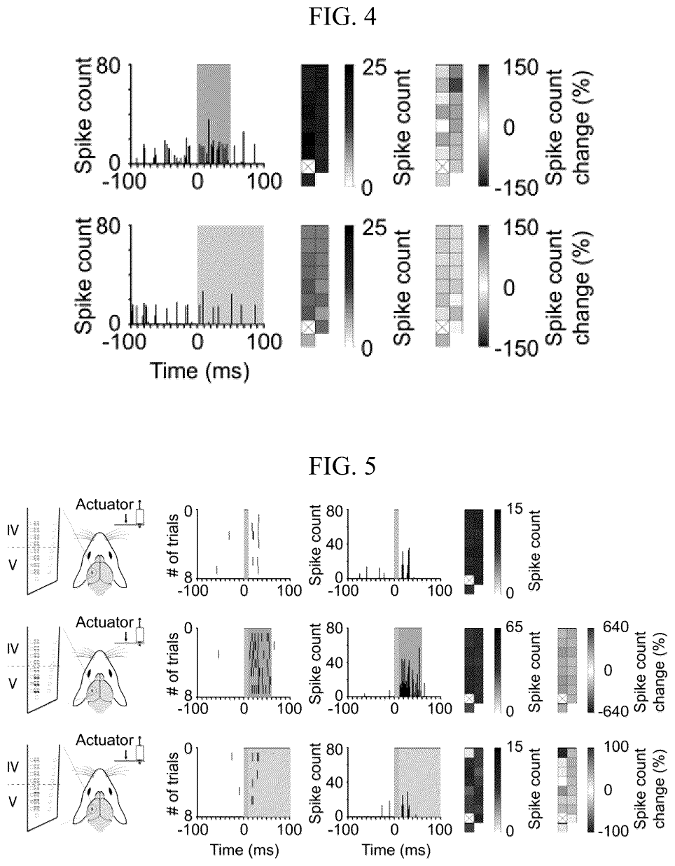

The data show that, when neurons were illuminated by 6 red LEDs, the spike count across the MEA was increased by 55±32% (top row in FIG. 4). This result may originate from additional depolarization of excitatory neurons via the red-light activation of Chrimson, which leads to more frequent spiking of their projected interneurons nearby during each red-LED pulse. On the other hand, when neurons were illuminated by 6 blue LEDs, the spike count across the MEA was decreased by 30±11% (bottom row in FIG. 4). This result may originate from additional hyperpolarization of excitatory neurons via the blue-light activation of GtACR2, which leads to less frequent spiking of their projected interneurons nearby during each odd-numbered blue LED pulse. In sum, the contrast between these two sets of data evidently suggests the capability of the probe for bi-directional in vivo optogenetic electrophysiology studies.

Layer-specific bi-directional optogenetic control over whisker-evoked neural activity

The probes were further evaluated to investigate if they can bi-directionally alter the sensory responses to whisker stimulation across layers IV and V. Here the work targeted to the whisker system as it is a preferred cortical model for mechanistic studies of sensory processing. Specifically, mouse whiskers were deflected contralateral to the brain region we inserted the probe by pulsing a current-driven solenoid actuator, and at the same time recorded the cortical responses via the MEA. Similar to methods described above, spike sorting was conducted (excluded the 4-ms of data centered at the rising and falling edge of each whisker pulse to assure artifact-free analysis), generated raster plots and PSTHs (data were aligned by whisker pulses, and counted spikes in each electrode. The data shows that whisker deflections readily evoked sensory responses across layers IV and V ([−100 ms, 0] vs [0, 100 ms] windows: 40 vs 157). The data is shown in FIG. 5, top row. This data set a baseline to analyze the following experiments when both whisker stimulation and LED illumination were applied.

After testing the sensory responses to whisker stimulation alone, whisker stimulation and LED illumination were examined together to investigate if dual-color LEDs can optogenetically modify whisker-evoked sensory responses in a bi-directional manner. To achieve this goal, 6 red LEDs positioned in layer V were pulsed (FIG. 5, middle row; seven 2-s periods with t_{off/on/off}=0.51/0.05/1.44 s), with each red LED pulse following right after a 10-ms whisker pulse (in this configuration, whisker stimulations are still offered in seven 2-s periods with t_{off/on/off}=0.5/0.01/1.49 s). To study the layer specificity, the same set of experiments were conducted by illuminating 6 red LEDs positioned in layer IV.

Following the same data analysis as described above, it was found that illuminating red LEDs positioned in either layer further enhanced the neural activity across layer IV and V compared to the whisker-stimulation-alone experiment, evidenced by an increase of the total spike count ([0, 100 ms] window from all 7 trials) across 16 electrodes (FIG. 5, top row). This result reaffirms the optogenetic excitation effect of red LEDs observed in the LED-only experiment. On the layer-specificity end, it was found that illuminating red LEDs in layer IV was more effective to enhance whisker-evoked neural activity than illuminating those in layer V (444±86% vs 266±37%). This result reaffirms the heterogeneity of cortical circuits and the need for high-resolution neuromodulation with close-packed micro-LEDs. Such layer specificity may relate to the signaling pathways from layer IV to layer V in the S1 region, where layer IV could act as the upstream of other cortical layers in forming the interlayer circuitry. If this were the case in our experiments, red-LED-illuminated excitatory neurons in layer IV may activate downstream interneurons (e.g., PV neurons) across both layers via synaptic projections, resulting in a stronger optogenetic excitation effect. Finally, it was observed that illuminating 3 red LEDs in either layer can sufficiently enhance whisker-evoked neural activity, suggesting that the close-packed LEDs can offer optogenetic control at high spatial resolution.

The optogenetic effect of blue LEDs on whisker-evoked neural activity in layers IV and V was also studied. Here, 6 blue LEDs positioned in either layer IV or layer V (FIG. 5, bottom row; seven 2-s periods with t_{off/on/off/on}=0.5/0.5/0.5/0.5 s) were pulsed, with each odd-numbered blue LED pulse started together with a 10-ms whisker pulse (in this configuration, whisker stimulations are still offered in seven 2-s periods with t_{off/on/off}=0.5/0.01/1.49 s).

Following the same data analysis as described above, it was found that illuminating blue LEDs positioned in either layer will overall inhibit the neural activity across layer IV and V compared to the whisker-stimulation-alone experiment, evidenced by a decrease of the total spike count ([0, 100 ms] window from all 7 trials) in the majority of 16 electrodes. This result reaffirms the optogenetic inhibition effect of blue LEDs observed in the LED-only experiment. On the layer-specificity end, it was noted that illuminating blue LEDs in layer V decreased the whisker-evoked spike count more uniformly across the MEA than illuminating those in layer IV (-26±22% vs −22±40%; their mean values were similar). This is likely because blue-LED-illuminated excitatory neurons in layer IV may inhibit interneurons across both layers (e.g., somatostatin-expressing (SST) neurons). Inhibiting these interneurons may effectively weaken the inhibition (and thus increase the activity) of their downstream interneurons (e.g., PV neurons), which may result in an increase of spike counts near some electrodes. Such effect could be less significant when layer V was illuminated by blue LEDs, since the optogenetic effect may influence fewer interneurons (largely located in layer V).

Thus the present inventor has developed a monolithic neural probe integrated with close-packed dual-color micro-LEDs and microelectrodes, aiming for high-resolution bi-directional optogenetic electrophysiology. Bright, localized, and fast-pulsed light from LED pixels, combined with low-impedance, low-artifact MEA, powers bi-directional in vivo optogenetic electrophysiology studies across cortical layers in anesthetized mice. Using the present probes, layer-specific bi-directional optogenetic control over whisker-evoked neural activity in layers IV and V was observed, which suggests the heterogeneity of cortical circuits and the need of high-precision neural interfacing with these close-packed dual-color LEDs.

Such bi-directional, interlayer optogenetic electrophysiology enabled by the present probe may add to high-resolution functional mapping of the brain circuitry, particularly those involving interlayer neurocircuits or highly heterogenous cortical regions. Leveraging their scalability, these probes can be tailor designed with suitable LED or MEA pitches/counts for targeted science problems, or extended into multi-shank neural probes for accessing more cortical areas. Besides cortex dynamics, the length of the present probes (ca. 5-mm) can also lend themselves for deep brain access. For instance, combining with population-specific opsin expression, the probes may offer bi-directional control over local circuits or individually excite two neuronal populations in the deep brain, which can help develop animal disease models at high precision (e.g., Parkinson, epilepsy).

A significant advantage is therefore provided by the present disclosure.

Experimental details follow.

### Probe Fabrication

A cross-barred blue LED array was fabricated on a commercial epitaxial GaN-on-Si wafer (Enkris Semiconductor) a Ni/Au (7/10 nm) current spreading layer onto the p-GaN to further reduce the contact resistance. Specifically, Ti/Al/Cr/Au layers (10/70/10/120 nm), an indium tin oxide layer (ITO, 120 nm), and Cr/Au layers (10/120 nm) were chosen to serve as n-, p-, and pad-contacts, respectively. After passivating the array with a PECVD-SiO₂ layer (ca. 200 nm), a SU8 layer (ca. 5 μm) was spin coated on top and soft baked at 95° C. for 10 mins.

On the other end, an AlGaInP-on-GaAs wafer (Powerway Wafer) was immersed in NH₄OH:H₂O₂=1:6 to separate AlGaInP epilayers (ca. 5.86 μm) from the GaAs substrate. This wet etching step completely removed the GaAs substrate (ca. 350 μm) and stopped at the Si-GaInP etching-stop layer (171 nm). A pipette was used to transfer these separated epilayers from the etchant to the DI water, rinse them 3 times with fresh DI water, and placed them on the soft-baked SU8 layer coated on the blue LED array with the c-GaP layer (p-contact of red LEDs) facing up. These epilayers were permanently bonded to SU8 by drying the device in the desiccator, cross-linking SU8 via UV exposure, and a hard-baking step (200° C. for 30 mins). A slow temperature ramping process (ca. 10° C./min) was applied to prevent the radical flow of the SU8 underneath the epilayers. Next, a cross-barred red LED array was fabricated on the epilayers using the similar approach as the blue LED array. Specifically, Cr/Au layers (40/120 nm), an ITO layer (120 nm), and Cr/Au layers (40/120 nm) were chosen to serve as n-, p-, and pad-contacts, respectively. The entire array was then passivated by PECVD-SiO2 (ca. 200 nm) and SU8 (ca. 5 μm) layers.

On top of the passivated dual-color LEDs, a Cr/Au-based (10/50 nm) MEA was fabricated with Cr/Pt/Cr/Au layers (10/50/10/120 nm) as pad-contacts. The resulting device was passivated by sputtered SiO₂ (ca. 10 nm) and spin-coated SU8 (ca. 5 μm) layers, followed by a wet etching step (diluted buffered oxide etchant, 1:50) to open the electrode and pad areas.

On the front side of a LED-MEA integrated device, a 250 μm-wide trench region surrounding the designed shank structure was formed by a series of RIE steps, which served to remove SiO₂/SU8/GaN layers in the trench (patterned by a 12 μm photoresist layer) and stopped at the Si substrate. We then applied a DRIE step to remove 80 μm thickness of Si in the trench, which helped to define the final thickness of the probe (ca. 50 μm) while leaving ca. 30 μm over-etch tolerance during the following back-side thinning step. Next, a photoresist layer (ca. 12 μm) was patterned on the back side of the device, followed by thinning down the Si substrate by ca. 750 μm using another DRIE step. This step formed a ca. 50 μm thick probe structure and separated the probe from the bulk of the wafer.

A custom-made PCB was soldered with cable connectors (Molex, product no. 5051102091) and stainless wires (A-M Systems, catalog no. 791900) to serve as the reference/ground electrodes (ca. 1 cm length of the PFA coating was removed). Lastly, we wire-bonded the probe onto the PCB and encapsulated the bonding wires with a UV-curable epoxy (Epoxies, Et 60-7159).

### Electroplating and MEA Characterization

A potentiostat (Gamry, Reference 600+) was used to sequentially electroplate PEDOT:PSSlayers on individual electrode, followed by EIS and CV tests with a three-electrode configuration.

### LED Characterization

A current bias was applied to each LED pixel by a SMU (Keysight B2902A), and measured its optical power by an optical power meter (Thorlabs PM100D). The wavelength correction was set to 462 nm [625 nm] for blue [red] pixels to match its dominant photoluminescence wavelength. The measured power was divided by the pixel area (49 μm²) to obtain the optical power density. The I-V characteristics, spatial profile of the output light, and pulsed switching for each LED pixel were measured by the SMU and/or an upright microscope (Nikon FN1).

### Artifact Characterization

Using two flat flexible cables (FFC, SAMTEC, FJH-20-D-31.75-4), LEDs and the MEA on the probe were connected to a LED driving circuit and an electrophysiology amplifier chip (RHD2164, Intan technologies), respectively. The former includes the SMU for biasing the LED pixel and a microcontroller (Arduino UNO, powered by Keysight E36312A) for pixel selection; the latter is wired to an Intan chip interface board for controlling the cell recording. The SMU and the microcontroller were programmed in the Matlab platform (Mathworks); the list sweep function in the SMU can output a half-cycle sinusoidal signal with a 50-ms period.

To quantify the recording artifacts, a packaged probe (together with its reference and ground electrodes) was immersed in the 1× DPBS (Thermofisher) solution. The select LEDs were pulsed and simultaneously collected the recording traces. The recording signals were sampled at 20-kHz and bandpass filtered at 250 Hz-10 kHz; the 60-Hz noise and de offset were removed by built-in filters of an Intan RHD USB interface software (Intan Technologies). The SMU output signal was also recorded by the Intan chip to synchronize the LED illumination patterns with the recording traces. The recording artifacts were quantified by the Vpp values in these traces.

### Animal Procedures

All animal procedures were approved by the Institutional Animal Care and Use Committee at the University of Massachusetts Amherst. Adult male and female wildtype C57BL/6J mice (2-3 months old, 21-30 g) were used for bi-directional in vivo optogenetic study. Mice were group housed before surgery with food and water available ad libitum on a reversed 12 h:12 h light-dark cycle. During stereotaxic surgery, mice received 1-2.5% isoflurane anesthesia during all procedures. The body temperature was maintained at 37° C. with a Deltaphase isothermal pad and insulator. Mice were administered subcutaneous injections of carprofen and cefazolin mg/kg. AAV5 BiPOLES virus (AAV5-CaMKII-somBiPOLES-mCerulean; Addgene #154948) was injected bilaterally in 500 nL volume at the site of the whisker barrel cortex63 (AP-1.0, ML +/−3.0 and DV −1.5). After surgery, mice were single-housed and allowed to incubate at least 3 weeks before experimentation.

To conduct in vivo experiments, the probe-bonded PCB was attached onto a micro positioner in a stereotaxic electrode holder to accurately insert the probe to the target brain region. The probe was zeroed at the brain surface after making the craniotomy and lowered with a speed of ˜60 μm/min. Reference and ground electrodes were placed under the skin touching the skull. The probe was inserted to a depth of ca. 888 μm from the brain surface (AP −1.4, ML +3.5).

Immediately after in vivo experiments, the anesthetized mouse (isoflurane 1-2.5%) was transcardially perfused using cold 0.9% saline followed by 10% neutral buffered formalin (VWR 16004-128). The brains were extracted and post-fixed in formalin overnight at 4° C., and then sunk in 20% sucrose for 24 h. Afterwards, the brains were frozen in isopentane and cut into 40-μm coronal sections using a Leica CM3050 S cryostat. The serial sections floated in 1× DPBS at 4° C. until mounted onto slides for imaging.

### Optogenetic Electrophysiology

During in vivo optogenetic electrophysiology studies, select red/blue LED pixels were pulsed via the SMU and simultaneously conducted neural recording via the Intan chip (see Artifact characterization). For experiments involving whisker stimulations, a solenoid actuator (uxcell) was used, with a copper film attached to the plunger, to mechanically deflect mouse whiskers by applying 12 V voltage pulses (toff/on/off=0.5/0.01/1.49 s) via the SMU. The actuator was set to lightly touch the whiskers in the ready state and deflect the whiskers with a 3-mm traveling range when receiving a voltage pulse. A typical recording session (using the Intan chip with the same configuration in artifact characterization) contains one or two 14-s LED pulsing periods, some of which were paired with whisker stimulation.

This disclosure further encompasses the following aspects.

Aspect 1: An optical neural probe comprising a probe body; a shank extending from the probe body to a tip; wherein the tip comprises a first plurality of light emitting diode pixels and a first plurality of recording electrodes, wherein the first plurality of light emitting diode pixels comprises a first light emitting diode and a second light emitting diode, wherein the first light emitting diode and the second light emitting diode emit a different color of light; wherein the first plurality of light emitting diode pixels are arranged in groups, wherein each light emitting diode pixel in a group has a center-to-center distance of 10 to 50 micrometers; and wherein each group of light emitting diode pixels has a center-to-center distance of 30 to 150 micrometers.

Aspect 2: The optical neural probe of aspect 1, wherein each group of light emitting diode pixels comprises one of the first light emitting diode and one of the second light emitting diode.

Aspect 3: The optical neural probe of aspect 1, wherein each group of light emitting diode pixels comprises two of the first light emitting diode and two of the second light emitting diode.

Aspect 4: The optical neural probe of aspect 3, wherein the first light emitting diode pixels and the second light emitting diode pixels are arranged in a grid pattern.

Aspect 5: The optical neural probe of any of aspects 1 to 4, wherein the recording electrodes are positioned adjacent to the plurality of light emitting diode pixels, preferably wherein the first plurality of light emitting diode pixels are surrounded recording electrodes.

Aspect 6: The optical neural probe of any of aspects 1 to 5, wherein the first light emitting diode pixel is a red light emitting diode pixel.

Aspect 7: The optical neural probe of any of aspects 1 to 6, wherein the second light emitting diode pixel is a blue light emitting diode pixel.

Aspect 8: The optical neural probe of any of aspects 1 to 7, wherein the shank is configured to locate the tip at a probing depth to target a neuron.

Aspect 9: The optical neural probe of any of aspects 1 to 8, wherein the first plurality of light emitting diode pixels span a distance of 300 to 1500 micrometers at the probe tip, preferably 300 to 450 micrometers.

Aspect 10: The optical neural probe of any of aspects 1 to 9, wherein the shank has a thickness of less than 100 micrometers, preferably a thickness of 40 to 80 micrometers; a width of 100 to 250 micrometers; and a length of 3 to 12 millimeters.

Aspect 11: The optical neural probe of any of aspects 1 to 10, further comprising a second plurality of light emitting diode pixels and a second plurality of recording electrodes, wherein the second plurality of light emitting diode pixels comprises a first light emitting diode and a second light emitting diode, wherein the first light emitting diode and the second light emitting diode emit a different color of light; wherein the second plurality of light emitting diode pixels are arranged in groups, wherein each light emitting diode pixel in a group has a center-to-center distance of 10 to 30 micrometers; wherein each group of light emitting diode pixels has a center-to-center distance of 30 to 70 micrometers; and wherein the second plurality of light emitting diode pixels and the second plurality of recording electrodes are positioned 1 to 2 millimeters away from the first plurality of light emitting diode pixels and the first plurality of recording electrodes.

Aspect 12: The optical neural probe of aspect 11, wherein the second plurality of light emitting diode pixels span a distance of 300 to 1500 micrometers at the probe tip, preferably 300 to 450 micrometers.

Aspect 13: The optical neural probe of any of aspects 1 to 12, wherein the probe body comprises electrical connections for electrical communication with a printed circuit board.

Aspect 14: The optical neural probe of any of aspects 1 to 13, wherein the optical neural probe further comprises a printed circuit board bound to the probe body, preferably wherein the printed circuit board is electrically connected to the light emitting diode pixels and the recording electrodes.

Aspect 15: The optical neural probe of any of aspects 1 to 14, further comprising a second shank extending from the probe body to a second tip, wherein the second tip comprises a third plurality of light emitting diode pixels and a third plurality of recording electrodes, wherein the third plurality of light emitting diode pixels comprises a third light emitting diode and a fourth light emitting diode, wherein the third light emitting diode and the fourth light emitting diode emit a different color of light; wherein each light emitting diode pixel within the third plurality of light emitting diode pixels has a center-to-center distance of 10 to 50 micrometers; and wherein the third plurality of light emitting diode pixels are arranged in groups, wherein each group of light emitting diode pixels has a center-to-center distance of 30 to 150 micrometers.

Aspect 16: The optical neural probe of any of aspects 1 to 15, wherein the shank comprises silicon or an alloy thereof.

Aspect 17: The optical neural probe of any of aspects 1 to 16, wherein the optical neural probe is made by a method comprising: contacting a first layer comprising a first light emitting diode precursor with a substrate; patterning the first light emitting diode precursor to provide a first light emitting diode pixel array; applying a first passivating layer to the first light emitting diode pixel array; contacting a second layer comprising a second light emitting diode precursor with the first passivating layer; patterning the second light emitting diode precursor to provide a second light emitting diode pixel array; applying a second passivating layer to the second light emitting diode pixel array; patterning a microelectrode array on the second passivating layer; applying a third passivating layer to the microelectrode array; and etching the substrate to provide the optical neural probe having preselected dimensions.

Aspect 18: A method for the manufacture of an optical neural probe comprising a probe body; a shank extending from the probe body to a tip; wherein the tip comprises a first plurality of light emitting diode pixels and a first plurality of recording electrodes, wherein the plurality of light emitting diode pixels comprises a first light emitting diode and a second light emitting diode, wherein the first light emitting diode and the second light emitting diode emit a different color of light; the method comprising: contacting a first layer comprising a first light emitting diode precursor with a substrate; patterning the first light emitting diode precursor to provide a first light emitting diode array; applying a first passivating layer to the first light emitting diode array; contacting a second layer comprising a second light emitting diode precursor with the first passivating layer; patterning the second light emitting diode precursor to provide a second light emitting diode array; applying a second passivating layer to the second light emitting diode array; patterning a microelectrode array on the second passivating layer; applying a third passivating layer to the microelectrode array; and etching the substrate to provide the optical neural probe having preselected dimensions.

Aspect 19: A method of stimulating target neurons expressing light responsive proteins in a tissue, the method comprising: inserting an optical neural probe into the tissue, wherein the optical neural probe is according to any of aspects 1 to 17 or made by the method of aspect 18; activating the optical neural probe to deliver light to stimulate the target neurons; and measuring neuronal activity with the recording electrodes in response to stimulating the target neurons.

Aspect 20: The method of aspect 19, wherein the tissue is of a living test subject such as an animal or human, or wherein the tissue is an organoid.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

It will be understood that when an element is referred to as being “on” another element or “in contact” with another element, it can be directly on the other element or intervening elements may be present therebetween, unless explicitly stated otherwise. In contrast, when an element is referred to as being “directly on” or “directly in contact with” another element, there are no intervening elements present.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. An optical neural probe comprising
a probe body;
a shank extending from the probe body to a tip;
wherein the tip comprises
a first plurality of light emitting diode pixels and a first plurality of recording electrodes,
wherein the first plurality of light emitting diode pixels comprises a first light emitting diode and a second light emitting diode, wherein the first light emitting diode and the second light emitting diode emit a different color of light;
wherein the first plurality of light emitting diode pixels are arranged in groups, wherein each light emitting diode pixel in a group has a center-to-center distance of 10 to 50 micrometers; and
wherein each group of light emitting diode pixels has a center-to-center distance of 30 to 150 micrometers.

2. The optical neural probe of claim 1, wherein each group of light emitting diode pixels comprises one of the first light emitting diode and one of the second light emitting diode.

3. The optical neural probe of claim 1, wherein each group of light emitting diode pixels comprises two of the first light emitting diode and two of the second light emitting diode.

4. The optical neural probe of claim 3, wherein the first light emitting diode pixels and the second light emitting diode pixels are arranged in a grid pattern.

5. The optical neural probe of claim 1, wherein the recording electrodes are positioned adjacent to the plurality of light emitting diode pixels.

6. The optical neural probe of claim 1, wherein the first light emitting diode pixel is a red light emitting diode pixel.

7. The optical neural probe of claim 1, wherein the second light emitting diode pixel is a blue light emitting diode pixel.

8. The optical neural probe of claim 1, wherein the shank is configured to locate the tip at a probing depth to target a neuron.

9. The optical neural probe of claim 1, wherein the first plurality of light emitting diode pixels span a distance of 300 to 1500 micrometers at the probe tip.

10. The optical neural probe of claim 1, wherein the shank has
a thickness of less than 100 micrometers;
a width of 100 to 250 micrometers; and
a length of 3 to 12 millimeters.

11. The optical neural probe of claim 1, further comprising a second plurality of light emitting diode pixels and a second plurality of recording electrodes,
wherein the second plurality of light emitting diode pixels comprises a first light emitting diode and a second light emitting diode, wherein the first light emitting diode and the second light emitting diode emit a different color of light;
wherein the second plurality of light emitting diode pixels are arranged in groups,
wherein each light emitting diode pixel in a group has a center-to-center distance of 10 to 30 micrometers;
wherein each group of light emitting diode pixels has a center-to-center distance of 30 to 70 micrometers; and
wherein the second plurality of light emitting diode pixels and the second plurality of recording electrodes are positioned 1 to 2 millimeters away from the first plurality of light emitting diode pixels and the first plurality of recording electrodes.

12. The optical neural probe of claim 11, wherein the second plurality of light emitting diode pixels span a distance of 300 to 1500 micrometers at the probe tip.

13. The optical neural probe of claim 1, wherein the probe body comprises electrical connections for electrical communication with a printed circuit board.

14. The optical neural probe of claim 1, wherein the optical neural probe further comprises a printed circuit board bound to the probe body.

15. The optical neural probe of claim 1, further comprising a second shank extending from the probe body to a second tip, wherein the second tip comprises a third plurality of light emitting diode pixels and a third plurality of recording electrodes,
wherein the third plurality of light emitting diode pixels comprises a third light emitting diode and a fourth light emitting diode, wherein the third light emitting diode and the fourth light emitting diode emit a different color of light;
wherein each light emitting diode pixel within the third plurality of light emitting diode pixels has a center-to-center distance of 10 to 50 micrometers; and
wherein the third plurality of light emitting diode pixels are arranged in groups, wherein each group of light emitting diode pixels has a center-to-center distance of 30 to 150 micrometers.

16. The optical neural probe of claim 1, wherein the shank comprises silicon or an alloy thereof.

17. The optical neural probe of claim 1, wherein the optical neural probe is made by a method comprising:
contacting a first layer comprising a first light emitting diode precursor with a substrate;
patterning the first light emitting diode precursor to provide a first light emitting diode pixel array;
applying a first passivating layer to the first light emitting diode pixel array;
contacting a second layer comprising a second light emitting diode precursor with the first passivating layer;
patterning the second light emitting diode precursor to provide a second light emitting diode pixel array;
applying a second passivating layer to the second light emitting diode pixel array;
patterning a microelectrode array on the second passivating layer;
applying a third passivating layer to the microelectrode array; and
etching the substrate to provide the optical neural probe having preselected dimensions.

18. A method for the manufacture of an optical neural probe comprising
a probe body;
a shank extending from the probe body to a tip;
wherein the tip comprises
a first plurality of light emitting diode pixels and a first plurality of recording electrodes,
wherein the plurality of light emitting diode pixels comprises a first light emitting diode and a second light emitting diode, wherein the first light emitting diode and the second light emitting diode emit a different color of light;
the method comprising:
contacting a first layer comprising a first light emitting diode precursor with a substrate;
patterning the first light emitting diode precursor to provide a first light emitting diode array;
applying a first passivating layer to the first light emitting diode array;
contacting a second layer comprising a second light emitting diode precursor with the first passivating layer;
patterning the second light emitting diode precursor to provide a second light emitting diode array;
applying a second passivating layer to the second light emitting diode array;
patterning a microelectrode array on the second passivating layer;
applying a third passivating layer to the microelectrode array; and
etching the substrate to provide the optical neural probe having preselected dimensions.

19. A method of stimulating target neurons expressing light responsive proteins in a tissue, the method comprising:
inserting an optical neural probe into the tissue, wherein the optical neural probe is according to claim 1;
activating the optical neural probe to deliver light to stimulate the target neurons; and
measuring neuronal activity with the recording electrodes in response to stimulating the target neurons.

20. The method of claim 19, wherein the tissue is of a living test subject such as an animal or human, or wherein the tissue is an organoid.
